(19) United States
(12) Patent Application Publication
(10) Pub. No.: US 2012/0016290 A1
(43) Pub. Date: Jan. 19, 2012

(54) LED-BASED SKIN TREATMENT SYSTEM AND METHOD

(75) Inventor: Hyun Soon SHIN, Daejeon (KR)

(73) Assignee: Electronics and Telecommunications Research Institute, Daejeon (KR)

(21) Appl. No.: 13/179,094

(22) Filed: Jul. 8, 2011

(30) Foreign Application Priority Data
Jul. 19, 2010 (KR) 10-2010-0069478

(51) Int. Cl. A61N 5/06

(52) U.S. Cl. 604/20; 607/88

(57) ABSTRACT

Provided are an LED-based skin treatment system capable of performing a skin treatment in consideration of individuals' respective skin properties and a reaction of skin to a change in a patient's emotional state by using an LED light source, and a method thereof. The LED-based skin treatment system includes a skin diagnosis unit ascertaining a patient's skin properties and an extent of a skin reaction to a change in a patient's emotional state by using multiple sensors, and diagnosing a skin lesion and an extent thereof and a skin lesion occurrence location; a skin treatment unit controlling an output state of an LED light source according to the skin lesion and the extent thereof and the skin lesion occurrence location; and a hardware control unit driving the multiple sensors and the LED light source.

### CROSS-REFERENCE TO RELATED APPLICATIONS

This application claims the priority of Korean Patent Application No. 10-2010-0069478 filed on Jul. 19, 2010, in the Korean Intellectual Property Office, the disclosure of which is incorporated herein by reference.

### FIELD OF THE INVENTION

The present invention relates to a skin treatment system and method, and more particularly, to a light emitting diode (LED) based skin treatment system capable of performing a skin treatment in consideration of individuals' respective skin properties and a reaction of skin to a change in a patient's emotional state by using an LED light source, and a method thereof.

### BACKGROUND OF THE INVENTION

### Description of the Related Art

Research into information technology (IT) convergence technologies for improved quality of life has recently been undertaken. In particular, skin ailments due to factors such as environmental pollution, stress, or the like, are increasing in contemporary society, and therefore, research into skin treatments has increased, while laser skin treatment therapies have increasingly appeared. However, the amount of emitted light energy in laser treatment therapies is able to affect and damage skin cells in a localized region of skin to thus destroy or eliminate affected areas. Since the laser treatment therapies have a relatively high level of skin stimulation, there may be a greater possibility of causing side effects. Furthermore, treatment costs are relatively high.

In addition, despite the fact that skin properties are different in respective individuals, identical treatment methods have been used, according to the related art, therefore, the identical treatments may provide different effects according to individuals to whom they are applied.

### SUMMARY OF INVENTION

An aspect of the present invention provides an LED-based skin treatment system using an LED light source instead of a laser, and a method thereof.

Another aspect of the present invention provides an LED-based skin treatment system able to provide a skin treatment appropriate for varied individuals while taking into account even a personal emotional change, and a method thereof.

According to an aspect of the present invention, there is provided an LED-based skin treatment system including: a skin diagnosis unit ascertaining a patient's skin properties and an extent of a skin reaction to a change in a patient's emotional state by using multiple sensors, and diagnosing a skin lesion, an extent thereof and a skin lesion occurrence location; a skin treatment unit controlling an output state of an LED light source according to the skin lesion, the extent thereof and the skin lesion occurrence location; and a hardware control unit driving the multiple sensors and the LED light source.

The LED light source may be implemented by a plurality of LEDs disposed in an array form.

The skin diagnosis unit may include: a skin property analyzer ascertaining the patient's skin properties; a skin reaction recognizer analyzing an extent of a skin reaction to a change in a patient's emotional state; and a skin disease definer defining the skin lesion and the extent thereof on the basis of the skin reaction extent provided by the skin properties and the emotional change of the patient.

The skin diagnosis unit may further include a treatment reaction sensor sensing a skin treatment extent to thus provide a result thereof to the skin treatment unit.

The skin treatment unit may include a light wavelength controller selecting a light wavelength according to the skin lesion; a light dimming controller controlling a light dimming level according to the skin lesion and the extent thereof; a light irradiation time controller controlling a light irradiation time according to the skin lesion and the extent thereof; a light amplitude controller controlling a light amplitude according to the skin lesion and the extent thereof; a light output controller controlling a light output so as to uniformly irradiate a light wavelength selected according to the skin lesion; a catalyst controller supplying a catalyst for accelerating an active oxygen state entry; and an active oxygen state controller controlling a light control state to maintain an active oxygen state.

The skin treatment unit may include a multi-wavelength controller diversifying types of generated light wavelengths in consideration of the skin lesion and the skin lesion occurrence location; a light source amplitude controller controlling a light amplitude according to the respective skin lesion occurrence locations; and a light output controller micro-adjusting the level of output light to respective skin regions having skin lesions occurring therein.

The skin treatment unit may further include a light frequency skin color coordinating controller controlling a frequency of color coordinated with skin cells; and a stable clinic controller stabilizing stimulated, tense skin.

The hardware controller may include a sensor control portion controlling each operation of the multiple sensors; a light source control portion controlling an operation of the LED light source; a light source module socket supporting various configurations and models of the LED light source; and a power control portion reducing an amount of power consumed by the LED light source.

The LED-based skin treatment system may further include a system control unit performing system management, as well as an interfacing operation between the hardware control unit and the skin treatment unit.

The system control unit may include a heat emission controller determining a heat emission extent of the system to stabilize a wavelength of the LED light source; a wide area controller determining a light irradiation area of light emitted from the LED light source and a density thereof; a super-precision controller determining a light collection extent and a light irradiation angle of the LED light source for the treatment of a local skin region; and a light source control interface portion providing an interface between the hardware control unit and the skin treatment unit.

In addition, the LED-based skin treatment system may further include a user input-output control unit sensing and acquiring a command input from a therapist to thus display a menu necessary for a system manipulation or information concerning a curing procedure.

The user input-output control unit may include a user authentication portion performing a user authentication procedure; a user input interface portion sensing and obtaining a command of a therapist; a menu control portion providing a menu necessary for system manipulation; and a treatment procedure output interface portion displaying information concerning a curing procedure.

According to another aspect of the present invention, there is provided an LED-based skin treatment method including: ascertaining skin properties and an extent of a skin reaction to a change in a patient's emotional state; ascertaining a presence of skin lesion, an extent thereof and a skin lesion occurrence location according to the skin properties and the skin reaction extent of a change in a patient's emotional state to thus control a light output state of an LED light source; supplying a catalyst; and maintaining an active oxygen state until a treatment is completed when skin reaches the active oxygen state.

### BRIEF DESCRIPTION OF THE DRAWINGS

The above and other aspects, features and other advantages of the present invention will be more clearly understood from the following detailed description taken in conjunction with the accompanying drawings, in which:

FIG. 1 is a schematic block diagram of an LED-based skin treatment system according to an embodiment of the present invention;

FIG. 2 is a detailed block diagram of an LED-based skin treatment system according to an embodiment of the present invention;

FIG. 3 illustrates an optical chemical reaction algorithm inducing an active oxygen state;

FIG. 4 is an operation concept diagram of an LED-based skin treatment system according to an embodiment of the present invention; and

FIG. 5 is a flowchart of a method of operating an LED-based skin treatment system according to an embodiment of the present invention.

### DETAILED DESCRIPTION OF THE INVENTION

The present invention may be modified variably and may have various embodiments, particular examples of which will be illustrated in drawings and described in detail.

However, it should be understood that the following exemplifying description of the invention is not intended to restrict the invention to specific forms of the present invention but rather the present invention is meant to cover all modifications, similarities and alternatives which are included in the spirit and scope of the present invention.

While terms such as “first” and “second,” etc., may be used to describe various components, such components must not be understood as being limited to the above terms. The above terms are used only to distinguish one component from another. For example, a first component may be referred to as a second component without departing from the scope of rights of the present invention, and likewise a second component may be referred to as a first component. The term “and/or” encompasses both combinations of the plurality of related items disclosed and any item from among the plurality of related items disclosed.

When a component is mentioned as being “connected” to or “accessing” another component, this may mean that it is directly connected to or accessing the other component, but it is to be understood that another component may exist therebetween. On the other hand, when a component is mentioned as being “directly connected” to or “directly accessing” another component, it is to be understood that there are no other components in-between.

The terms used in the present application are merely used to describe particular embodiments, and are not intended to limit the present invention. An expression used in the singular encompasses the expression of the plural, unless it has a clearly different meaning in the context in which it is used. In the present application, it is to be understood that terms such as “including” or “having,” etc., are intended to indicate the existence of features, numbers, operations, actions, components, parts, or combinations thereof disclosed in the specification, and are not intended to preclude the possibility that one or more other features, numbers, operations, actions, components, parts, or combinations thereof may exist or may be added.

Unless otherwise defined, all terms used herein, including technical or scientific terms, have the same meanings as those generally understood by those with ordinary knowledge in the field of art to which the present invention belongs. Such terms as those defined in a generally used dictionary are to be interpreted as having meanings equal to the contextual meanings in the relevant field of art, and are not to be interpreted as having ideal or excessively formal meanings unless clearly defined as having such in the present application.

Embodiments of the present invention will be described below in detail with reference to the accompanying drawings, where those components that are the same or are in correspondence are referred to by using the same reference number, regardless of the figure number, and redundant explanations are omitted.

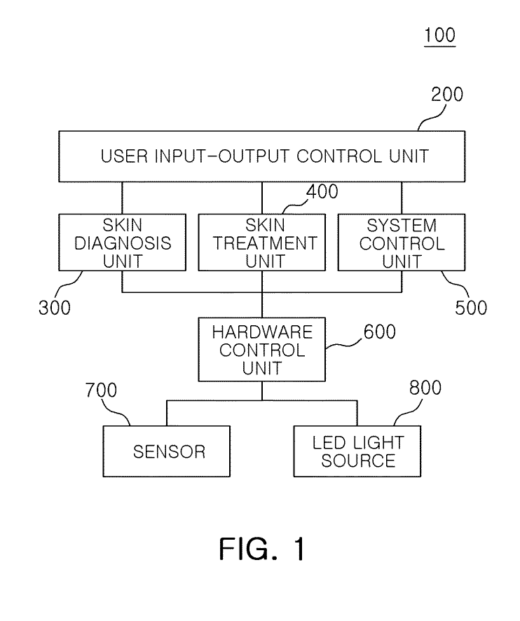

FIG. 1 is a schematic block diagram of an LED-based skin treatment system according to an embodiment of the present invention.

Referring to FIG. 1, a skin treatment system 100 according to an embodiment of the present invention may largely include a user input-output control unit 200, a skin diagnosis unit 300, a skin treatment unit 400, a system control unit 500, a hardware control unit 600, multiple sensors 700, and an LED light source 800.

The user input-output control unit 200 may sense and acquire a command input from a therapist to thus display a menu necessary for system manipulation, various types of information relating to a current treatment process, or the like.

The skin diagnosis unit 300 may ascertain a patients' skin properties, for example, an oil/moisture extent, a skin thickness and dead skin cell buildup, an amount of melanin pigment, a collagen component, an elastin component or the like, and an extent of a skin reaction to a change in a patient's emotional state. Further, on the basis of the ascertainment results, the skin diagnosis unit 300 may define a skin lesion such as a pimple, atopic skin, whitening, aging, an extent thereof, for example, high, medium and low levels thereof and a skin lesion occurrence location (for example, forehead, cheek, chin, or the like).

The skin treatment unit 400 may perform a skin treatment using the LED light source 800 while controlling a light wavelength and/or color of light emitted from the LED light source 800, a light irradiation time, light strength, a light irradiation region, light irradiation sensitivity or the like on the basis of a diagnosis result from the skin diagnosis unit 300.

The system control unit 500 may be in charge of a management of the system, and an interface control between the skin treatment unit 400 and the hardware control unit 600.

The hardware control unit 600 may substantially drive the multiple sensors 700 and the LED light source 800.

The multiple sensors 700 may measure skin properties such as a patient's skin oil or/and moisture-content, thickness and dead skin cell buildup, a melanin amount, a collagen component, an elastin component, or an extent of a skin reaction to a change in a patient's emotional state, and may generate a sensing signal having a signal value corresponding thereto, to then provide the sensing signal to the skin diagnosis unit 300. In addition, the multiple sensors 700 may acquire and analyze a biosignal for ascertaining a patient's current emotional state such as a pulse rate, a skin resistance response, skin temperature, or the like, to then provide the analyzed biosignal to the skin diagnosis unit 30.

The LED light source 800 may irradiate light while variously controlling a light wavelength and/or color of light emitted from the LED light source 800, a light irradiation time, light strength, a localized region of a skin to receive light irradiation, light irradiation sensitivity, or the like, under a control of the skin treatment unit 400. Here, the LED light source 800 may be implemented by a plurality of LEDs disposed in an array form.

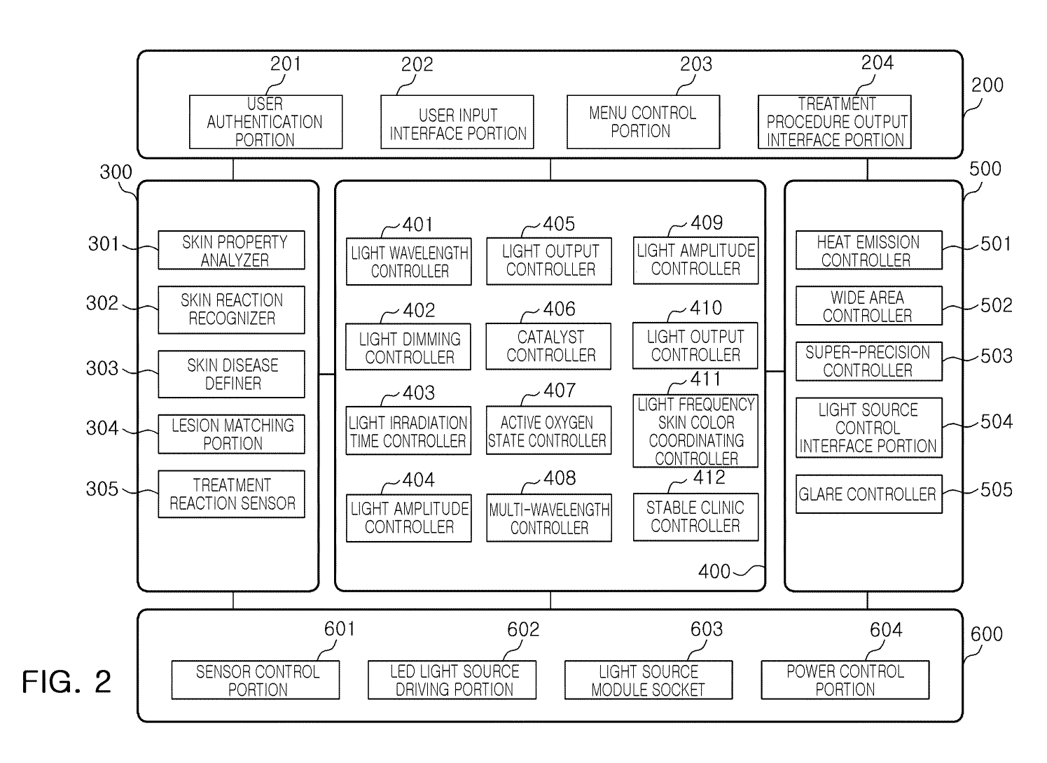

FIG. 2 is a detailed block diagram of an LED-based skin treatment system according to an embodiment of the present invention.

With reference to FIG. 2, the user input-output control unit 200 may include a user authentication portion 201 provided so that only a user authorized through a user authentication procedure can use the skin treatment system 100; a user input interface portion 202 sensing and obtaining a command from a therapist; a menu control portion 203 providing a menu necessary for system manipulation; and a treatment procedure output interface portion 204 displaying various types of information concerning a current curing procedure such that the therapist or patient can see the current curing procedure.

The skin diagnosis unit 300 may include a skin property analyzer 301, a skin reaction recognizer 302, a skin disease definer 303, a lesion matching portion 304, and a treatment reaction sensor 305. The skin property analyzer 301 may acquire and analyze sensing signals providing a patient's skin properties to thus ascertain the patient's skin properties. The skin reaction recognizer 302 may receive and analyze a biosignal to ascertain a change in a patient's emotional state to thus ascertain a patient's emotional quotient, and may then receive and analyze sensing signals determining an extent of the patient's skin reaction to thereby ascertain an extent of a skin reaction to a change in the patient's emotional state. The skin disease definer 303 may define a skin lesion extent on the basis of the patient's skin properties and the skin reaction extent of a change in the patient's emotional state. The lesion matching portion 304 may define a skin lesion on the basis of the patient's skin properties and the extent of skin reaction provided by the patient's change in emotional state. The treatment reaction sensor 305 may ascertain an extent of a skin reaction such as skin temperature, an amount of oil/moisture on skin, a skin thickness and dead skin cell buildup, an amount of melanin, a collagen component, an elastin component, or the like, during a skin curing procedure, to thus feed a result thereof back to the skin treatment unit 400 such that the skin treatment unit 400 may precisely control a light output.

The skin treatment unit 400 may include a light wavelength controller 401, a light dimming controller 402, a light irradiation time controller 403, a light amplitude controller 404, a light output controller 405, a catalyst controller 406, and an active oxygen state controller 407. The light wavelength controller 401 may select a light wavelength appropriate for a skin lesion. The light dimming controller 402 may control a light dimming level on the basis of the skin lesion and the extent thereof, in order to control a level of light irradiation emitted from a light source at a relatively precise level. The light irradiation time controller 403 may control a light irradiation time on the basis of the skin lesion and the extent thereof. The light amplitude controller 404 may control a light amplitude on the basis of the skin lesion and the extent thereof. The light output controller 405 may control a light output so as to uniformly irradiate a light wavelength selected by the wavelength controller 401. The catalyst controller 406 may supply a catalyst for accelerating an active oxygen state entry. The active oxygen state controller 407 may control a light control state to maintain an active oxygen state.

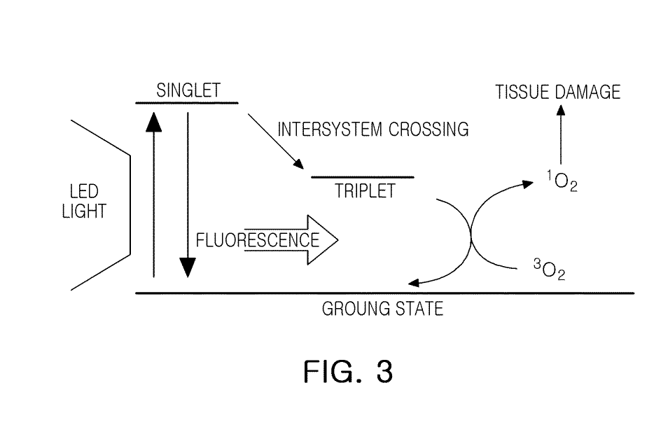

For reference, an active oxygen state indicates a state in which a large quantity of active oxygen such as oxygen or peroxide is generated to remove a pathogen or a foreign body according to an optical chemical reaction as shown in FIG. 3. According to the embodiment of the present invention, the active oxygen state may be artificially generated to thereby cure skin diseases by using a principle of activating and reproducing skin cells through the active oxygen production state.

In the case of necessity, the skin treatment unit 400 may consider a skin lesion occurrence location as well as the skin lesion and the extent thereof. The skin treatment unit 400 may further include a multi-wavelength controller 408 diversifying types of generated light wavelengths according to the skin lesion and the skin lesion occurrence locations, a light amplitude controller 409 diversifying types of light amplitudes according to respective skin lesion occurrence locations, and a light output controller 410 micro-adjusting a level of output light according to the respective skin lesion occurrence locations.

In addition, the skin treatment unit 400 may further include a light frequency skin color coordinating controller 411 controlling a frequency of light to be coordinated with skin color in order to speed up the metabolism of skin cells, and a stable clinic controller 412 stabilizing tense, stimulated skin after the treatment.

The system control unit 500 may include a heat emission controller 501, a wide area controller 502, a super-precision controller 503, a light source control interface portion 504, and a glare controller 505. The heat emission controller 501 may determine a heat emission extent of the system to stabilize a light wavelength for treatment. The wide area controller 502 may determine a light irradiation area of light emitted from the LED light source and a density thereof in consideration of skin lesions and skin lesion occurrence locations, that is, turning respective LEDs on or turning each LED off according to the light irradiation area and the density thereof. The super-precision controller 503 may control a light collection extent and a light irradiation angle of the LED light source for the treatment of a local skin region. The light source control interface portion 504 may perform an interfacing operation between the hardware control unit 600 and the skin treatment unit 400 to support a control of the light source. The glare controller 505 may prevent a glaring phenomenon from affecting a patient.

The hardware controller 600 may include a sensor control portion 601 controlling each operation of the multiple sensors 700 and receiving a sensing signal provided from the multiple sensors 700 to thus provide the received sensing signal to the skin diagnosis unit 300, an LED light source driving portion 602 actually driving the LED light source 800, a light source module socket 603 supporting a configuration of the LED light source 800 and an extension of a model and a change thereof, and a power control portion 604 supporting a power reduction of the LED light source 800.

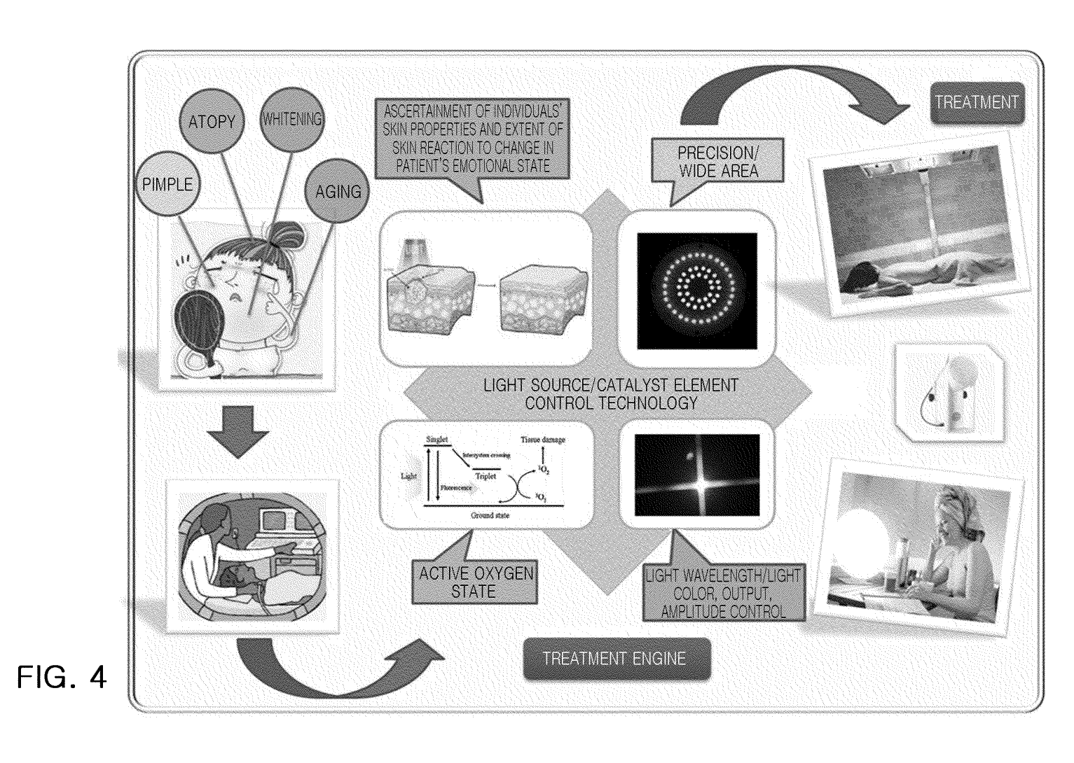

FIG. 4 is an operation concept diagram of an LED-based skin treatment system according to an embodiment of the present invention.

As shown in FIG. 4, the skin treatment system may diagnose skin properties such as whitening, atopy and aging according to respective individuals, and according to the diagnosis results, the treatment system may control a light wavelength/color of light emitted from the LED light source, an output thereof, an amplitude thereof, or the like, and may also control precision in the LED light source and a light irradiation area thereof and a density thereof, or the like, thereby generating an active oxygen state in the skin.

Whereby, an active oxygen state may be induced using the LED light source to thereby promote a treatment reaction according to the embodiment of the present invention, instead of performing a treatment by destroying an affected skin area by using a laser.

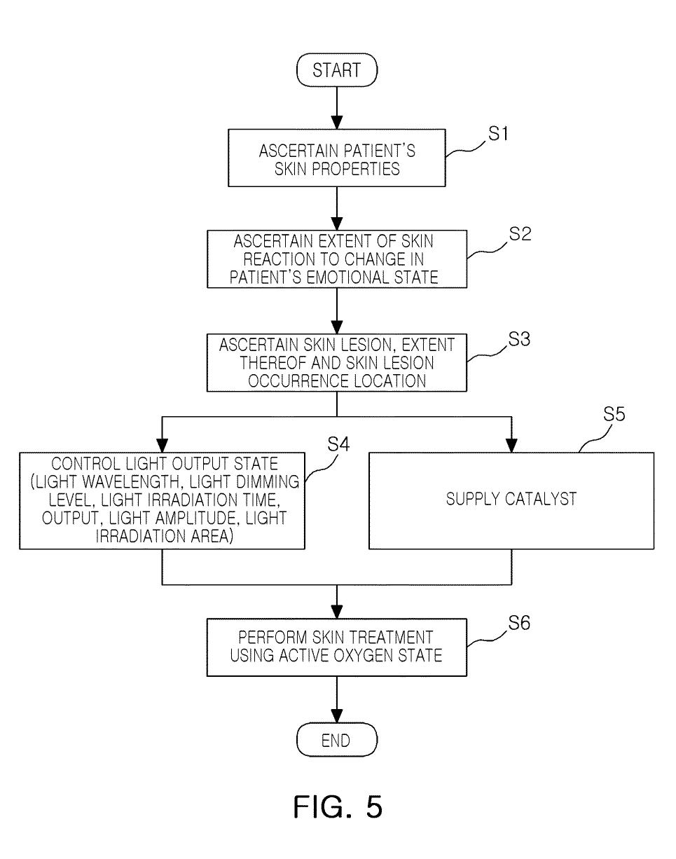

FIG. 5 is a flowchart of a method of operating an LED-based skin treatment system according to an embodiment of the present invention.

With reference to FIG. 5, first, in operation S1, the multiple sensors may sense an oil/moisture extent, a skin thickness and dead skin cell buildup, an amount of melanin pigment, a collagen component and an elastin component in the skin of a patient, to thus ascertain a patient's skin properties on the basis of the sensed results.

Then, a patient's current emotional state may be ascertained by acquiring and analyzing the biosignal that denotes the patient current emotional state such as a patient's pulse rate, a skin resistance response, skin temperature, and the like, to thus ascertain the patient's current emotional state and an extent of a skin reaction thereto through the multiple sensors. That is, the extent of a skin reaction to a change in the patient's emotional state may be obtained in operation S2.

Subsequently, a skin lesion and an extent thereof, and a skin lesion occurrence location may be ascertained on the basis of the patient's skin properties and the change in the patient's emotional state obtained in operations S1 and S2, in operation S3.

Then, on the basis of the skin lesion, the extent thereof and the skin lesion occurrence locations, a light output state may be controlled in operation S4.

In operation 4, in order to substantially increase treatment effects, simultaneously or in sequence, operations are performed, for example, a skin lesion-based light wavelength selection, a light dimming level control based on a skin lesion and an extent thereof, a light irradiation time based on a skin lesion and an extent thereof, a light amplitude control based on a skin lesion and an extent thereof, a skin lesion-based light operation control, a multi-wavelength control for substantially increasing skin disease treatment effects, a light amplitude type diversification to respective skin lesion occurrence locations, an light output diversification with regard to respective skin lesion occurrence locations, and the like.

Moreover, in order to heighten a therapist's convenience and treatment efficiency, operations in which a system heat emission control operation for stabilizing an output state from the LED light source, for example, a maintenance of a constant light wavelength and output, a wide area and selective optical mode (rectangular, saw, bunch, or the like) control operation (for example, a light irradiation area and density controlling operation) according to skin lesion occurrence locations and lesion models, a light collection extent and light irradiation angle controlling operation, and the like, may be performed at the same time or in sequence.

Simultaneously with performing operation S4, a catalyst accelerating an active oxygen state may be supplied such that skin cells are activated and reproduced using a chemical reaction in the skin cells, in operation S5.

When the skin reaches the active oxygen state through the light output control procedure of operation S4 and the catalyst supply procedure of S5, a control operation of stabilizing the active oxygen state to maintain the active oxygen state may be performed in operation S6 until the treatment is completed.

Although not shown in FIG. 5, when the treatment using the LED light source is completed, a stable clinic procedure may be additionally performed to stabilize tense, stimulated skin due to the treatment using the LED light source.

As set forth above, in the LED-based skin treatment system and method according to embodiments of the present invention, skin cells may be activated and reproduced through an optical chemical reaction in the skin cells by using the LED light source, to thereby treat skin diseases, unlike laser treatment therapies through a skin destroy and incision using a laser. Therefore, a treatment region may be diversified with a relatively wider range since it may be applied to a large quantity of skin cells. In addition, a stable treatment and relatively low treatment costs may be achieved instead of those in a laser skin treatment.

In addition, in the LED-based skin treatment system and method according to the embodiments of the present invention, patients' respective skin properties and an extent of a skin reaction to a change in a patient's emotional state may be ascertained, and on the basis of the ascertained results, an optical control operation for a skin treatment maybe executed. Whereby, a relatively more optimized skin treatment for respective patients may be attained.

While the present invention has been shown and described in connection with the embodiments in the, it will be apparent to those skilled in the art that modifications and variations can be made without departing from the spirit and scope of the invention as defined by the appended claims.

What is claimed is:

1. A light emitting diode (LED) based skin treatment system, comprising:
a skin diagnosis unit ascertaining a patient's skin properties and an extent of a skin reaction to a change in a patient's emotional state by using multiple sensors, and diagnosing a skin lesion and an extent thereof and a skin lesion occurrence location;
a skin treatment unit controlling an output state of an LED light source according to the skin lesion and the extent thereof and the skin lesion occurrence location; and
a hardware control unit driving the multiple sensors and the LED light source.

2. The system of claim 1, wherein the LED light source is implemented by a plurality of LEDs disposed in an array form.

3. The system of claim 1, wherein the skin diagnosis unit includes:
a skin property analyzer ascertaining the patient's skin properties;
a skin reaction recognizer analyzing an extent of a skin reaction to the change in the patient's emotional state; and
a skin disease definer defining the skin lesion and the extent thereof on the basis of the skin properties and the skin reaction extent to the change in the patient's emotional state.

4. The system of claim 3, wherein the skin diagnosis unit further includes a treatment reaction sensor sensing a skin treatment extent to thus provide a result thereof to the skin treatment unit.

5. The system of claim 3, wherein the skin treatment unit includes:
a light wavelength controller selecting a light wavelength according to the skin lesion;
a light dimming controller controlling a light dimming level according to the skin lesion and the extent thereof;
a light irradiation time controller controlling a light irradiation time according to the skin lesion and the extent thereof;
a light amplitude controller controlling a light amplitude according to the skin lesion and the extent thereof;
a light output controller controlling a light output so as to uniformly irradiate a light wavelength selected by the light wavelength controller;
a catalyst controller supplying a catalyst for accelerating an active oxygen state entry; and
an active oxygen state controller controlling a light control state to maintain an active oxygen state.

6. The system of claim 5, wherein the skin treatment unit includes:
a multi-wavelength controller diversifying types of generated light wavelengths in consideration of the skin lesion and the skin lesion occurrence location;
a light source amplitude controller controlling a light amplitude according to the respective skin lesion occurrence locations; and
a light output controller micro-adjusting the level of output light according to respective skin regions having skin diseases occurring therein even in the case of the same kinds of skin diseases.

7. The system of claim 5, wherein skin treatment unit further includes:
a light frequency skin color coordinating controller controlling a frequency of color coordinated with skin cells; and
a stable clinic controller stabilizing stimulated, tense skin.

8. The system of claim 6, wherein skin treatment unit further includes:
a light frequency skin color coordinating controller controlling a frequency of color coordinated with skin cells; and
a stable clinic controller stabilizing stimulated, tense skin.

9. The system of claim 1, wherein the hardware controller includes:
a sensor control portion controlling each operation of the multiple sensors;
a light source control portion controlling an operation of the LED light source;
a light source module socket supporting various configurations and models of the LED light source; and
a power control portion reducing an amount of power consumed by the LED light source.

10. The system of claim 1, further comprising a system control unit performing system management, as well as an interfacing operation between the hardware control unit and the skin treatment unit.

11. The system of claim 10, wherein the system control unit includes:
a heat emission controller determining a heat emission extent of the system to stabilize a wavelength of the LED light source;
a wide area controller determining a light irradiation area of light emitted from the LED light source and a density thereof;
a super-precision controller determining a light collection extent and a light irradiation angle of the LED light source for the treatment of a local skin region; and
a light source control interface portion providing an interface between the hardware control unit and the skin treatment unit.

12. The system of claim 1, further comprising a user input-output control unit sensing and acquiring a command input from a therapist to thus display a menu necessary for a system manipulation or information concerning a curing procedure.

13. The system of claim 12, wherein the user input-output control unit includes:
a user authentication portion performing a user authentication procedure;
a user input interface portion sensing and obtaining a command of a therapist;
a menu control portion providing the menu necessary for the system manipulation; and
a treatment procedure output interface portion displaying information concerning a curing procedure.

14. An LED-based skin treatment method, comprising:
ascertaining a patient's skin properties and an extent of a skin reaction to a change in a patient's emotional state;
ascertaining a presence of skin lesion, an extent thereof and a skin lesion occurrence location according to the patient's skin properties and the skin reaction extent to the change in the patient's emotional state, to thus control a light output state of an LED light source;
supplying a catalyst; and
maintaining an active oxygen state until a treatment is completed when skin reaches the active oxygen state.
